(19) United States
(12) Patent Application Publication
(10) Pub. No.: US 2012/0283683 A1
(43) Pub. Date: Nov. 8, 2012

(54) Diaper

(76) Inventors: Jung-Chi Tai, Tainan City (TW); Ho-Hsi Yang, Tainan City (TW); Chien-Chung Su, Tainan City (TW)

(21) Appl. No.: 13/239,718

(22) Filed: Sep. 22, 2011

(30) Foreign Application Priority Data
May 4, 2011 (TW) 100115590

(51) Int. Cl. A61F 13/49

(52) U.S. Cl. 604/385.3

(57) ABSTRACT

A diaper includes first and second sheets having elastic strings, and a crotch section with an absorbent body connected to the first and second sheets. The first sheet includes two first margins, each of which is contiguous to one of first outermost edges, and a first notch that is indented inwardly from one of the first outermost edges to extend inwardly through and beyond the adjacent first margin. The second sheet includes two second outermost edges, two second margins. The second margins are thermal bonded respectively to the first margins to form thermal seams. The first notch forms a first opening contiguous to one of the thermal seams to provide good ventilation and to ease tearing of the diaper at the thermal seam.

### CROSS REFERENCE TO RELATED APPLICATION

This application claims priority of Taiwanese application no. 100115590 filed on May 4, 2011.

### BACKGROUND OF THE INVENTION

1. Field of the Invention

The invention relates to a diaper, more particularly to a diaper with good air permeability.

2. Description of the Related Art

Referring to FIG. 1, a conventional diaper 9 comprises a waist-surrounding part 91 surrounding a user's waist and a crotch section having an absorbent body 92. The waist-surrounding part 91 is elastic and can provide a restraining force to prevent the diaper 9 from sliding down. The absorbent body 92 is used to absorb urine and feces. In use, two opposite lateral edges 911, 912 of the waist-surrounding part 91 may be torn apart to take off or replace the diaper. However, because the waist-surrounding part 91 of the conventional diaper 9 is formed by thermal bonding the lateral edges 911, 912 of two longitudinal sheets 913, 914, the sheets 913, 914 cannot be easily torn apart along the thermal bonding lines thereof. Since an operator has to apply a substantial force to take off the diaper, the urine and/or feces on the diaper may stain other body parts of the wearer, or even the body of the operator. Moreover, the waist-surrounding part 91, which usually incorporates a resilient material, such as elastic strings 93, or elastic bands, is rarely air permeable, and is likely to produce skin diseases such as eczema, on the wearer's skin.

### SUMMARY OF THE INVENTION

An object of the present invention is to provide a diaper that can be easily torn apart along thermal bonding lines and that has good air permeability.

Accordingly, the present invention provides a diaper which comprises a first sheet, a second sheet, elastic strings disposed in the first and second sheets, and a crotch section having an absorbent body and connected to the first and second sheets. The first sheet is longitudinal and includes two first outermost edges that are opposite in the longitudinal direction of the first sheet, two first margins, each of which is contiguous to one of the first outermost edges, and at least one first notch that is indented inwardly from one of the first outermost edges to extend inwardly through and beyond an adjacent one of the first margins. The first notch has a dimension in the longitudinal direction, which is larger than that of the adjacent one of the first margins. The second sheet is longitudinal and includes two second outermost edges that are opposite in the longitudinal direction of the second sheet, and two second margins, each of which is contiguous to one of the second outermost edges and each of which is in face-to-face contact with one of the first margins. The second margins are thermal bonded respectively to the first margins to form thermal seams so that the first and second sheets are formed into a waist-surrounding part. The first notch forms a first opening contiguous to one of the thermal seams.

### BRIEF DESCRIPTION OF THE DRAWINGS

Other features and advantages of the present invention will become apparent in the following detailed description of the preferred embodiments with reference to the accompanying drawings, of which:

FIG. 1 is a perspective view of a conventional diaper;

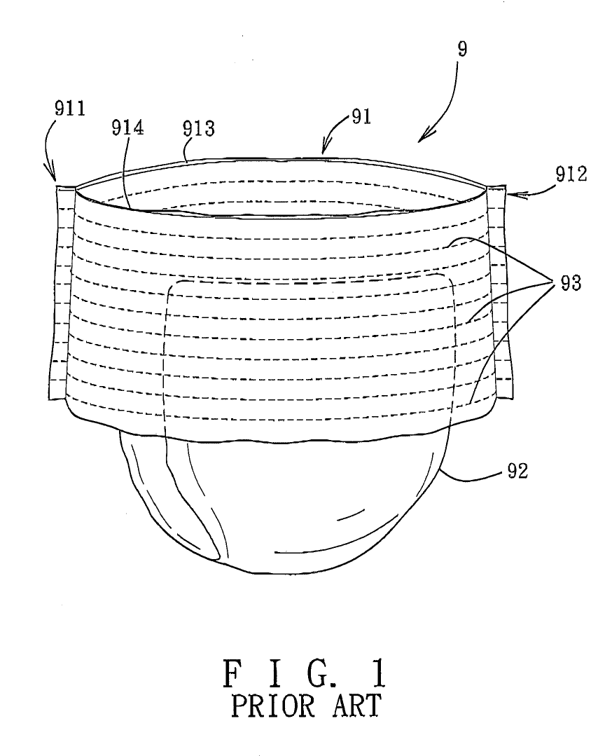

FIG. 2 is a schematic plan view of the first preferred embodiment of a diaper according to the present invention;

FIG. 3 is a perspective view of the first preferred embodiment;

FIG. 4 is a schematic plan view of the second preferred embodiment of a diaper according to the present invention;

FIG. 5 is a schematic plan view of the third preferred embodiment of a diaper according to the present invention;

FIG. 6 is a schematic plan view of the fourth preferred embodiment of a diaper according to the present invention; and

FIG. 7 is a schematic plan view of the fifth preferred embodiment of a diaper according to the present invention.

### DETAILED DESCRIPTION OF THE PREFERRED EMBODIMENTS

Before the present invention is described in greater detail with reference to the accompanying preferred embodiments, it should be noted herein that like elements are denoted by the same reference numerals throughout the disclosure.

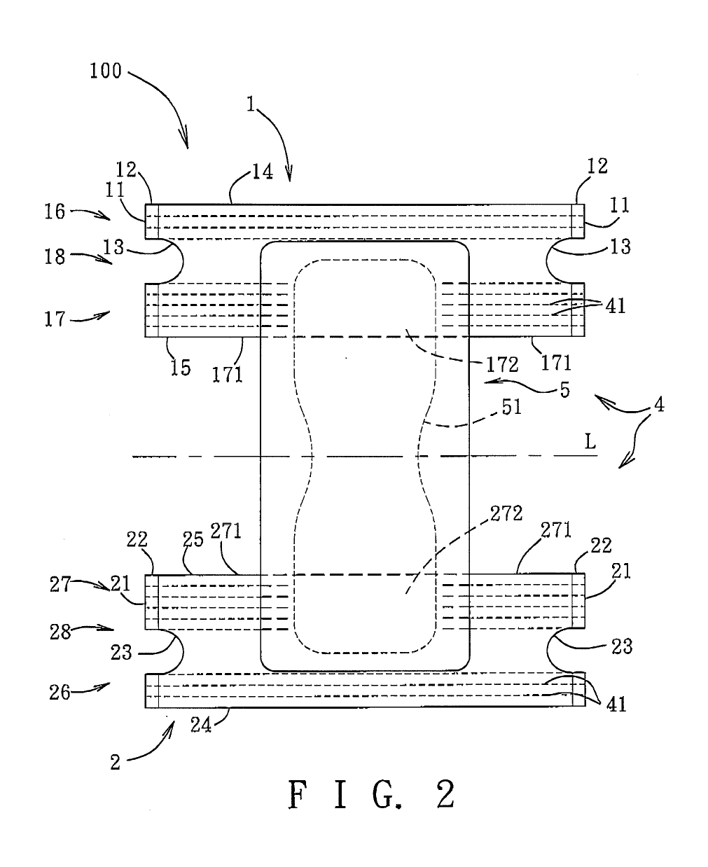

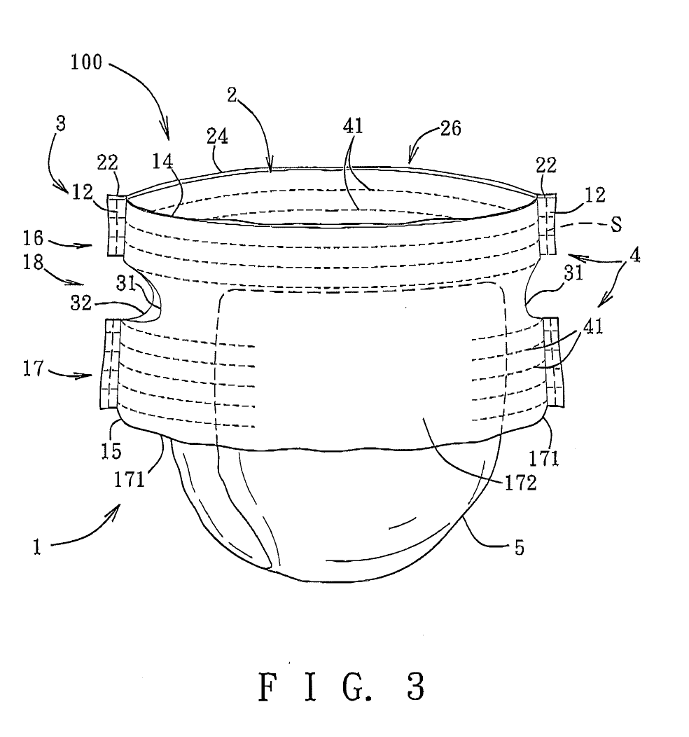

Referring to FIGS. 2 and 3, the first preferred embodiment of a diaper 100 according to the present invention is shown to include a first sheet 1, a second sheet 2, an elastic unit 4 and a crotch section 5. The first and second sheets 1, 2 are made of heat fusible material, such as plastic, so that the first and second sheets 1, 2 can be bonded together by thermal bonding.

The first sheet 1 is longitudinal and includes two first outermost edges 11 that are opposite in the longitudinal direction of the first sheet 1, two first margins 12, each of which is contiguous to one of the first outermost edges 11, two first notches 13, each of which is indented inwardly from a respective one of the first outermost edges 11 to extend inwardly through and beyond an adjacent one of the first margins 12, and first and second lateral edges 14, 15 disposed oppositely and interconnecting the first outermost edges 11. Each of the first notches 13 has a dimension in the longitudinal direction, which is larger than that of the adjacent one of the first margins 12. The first sheet 1 further includes a first area 16 adjacent to the first lateral edge 14, a second area 17 adjacent to the second lateral edge 15, and a third area 18 interposed between and contiguous to the first and second areas 16, 17. The second area 17 of the first sheet 1 is divided into two lateral regions 171 respectively contiguous to the first outermost edges 11, and a middle region 172 disposed between and contiguous to the lateral regions 171. The first notches 13 are disposed in the third area 18.

The second sheet 2 is longitudinal and includes two second outermost edges 21 that are opposite in the longitudinal direction of the second sheet 2, two second margins 22, each of which is contiguous to one of the second outermost edges 21, two second notches 23, each of which is indented inwardly from a respective one of the second outermost edges 21 to extend inwardly through and beyond an adjacent one of the second margins 22, and third and fourth lateral edges 24, 25 disposed oppositely and interconnecting the second outermost edges 21. Each of the second notches 23 has a dimension in the longitudinal direction, which is larger than that of the adjacent one of the second margins 22. The second sheet 2 further includes a fourth area 26 adjacent to the third lateral edge 24, a fifth area 27 adjacent to the fourth lateral edge 25, and a sixth area 28 interposed between and contiguous to the fourth and fifth areas 26, 27. The fifth area 27 of the second sheet 2 is divided into two lateral regions 271 respectively contiguous to the second outermost edges 21, and a middle region 272 disposed between and contiguous to the lateral regions 271. The second notches 23 are disposed in the sixth area 28.

The second margins 22 are bonded respectively to the first margins 12 by thermal bonding to form thermal seams (S) so that the first sheet 1 and the second sheet 2, when expanded, form a waist-surrounding part 3 to surround a user's waist. Through the provision of the first notches 13 and the second notches 23, the bonding areas at the thermal seams (S) is reduced, lowering the bonding strength between the first margins 12 and the second margins 22. An operator who takes care of a wearer can easily tear apart the waist-surrounding part 3 along the thermal seams (S). Moreover, because the first notches 13 extend inwardly beyond the respective first margins 12, two first openings 31 are formed in the waist-surrounding part 3, and because the second notches 23 extend inwardly beyond the respective second margins 22, two second openings 32 are formed in the waist-surrounding part 3. The first and second openings 31, 32 provide good ventilation for the waist-surrounding part 3. In this embodiment, the second notches 23 are aligned with the respective first notches 13 so that the first openings 31 are in spatial communication with the second openings 32.

The elastic unit 4 is provided in the first and second sheets 1, 2 to prevent the waist-surrounding part 3 from slipping. In this embodiment, the elastic unit 4 includes a plurality of spaced apart elastic strings 41 mounted along the longitudinal directions of the first sheet 1 and the second sheet 2. The elastic strings 41 are mounted on the first area 16, the lateral regions 171 of the second area 17, the fourth area 26, and the lateral regions 271 of the fifth area 27. The third area 18, the sixth area 28, the middle regions 172 of the second area 17 and the middle regions 271 of the fifth area 27 are free of the elastic strings 41. Therefore, the elastic strings 41 disposed on the first area 16 and the fourth area 26 provide a main constraining force, and those disposed on the lateral regions 171, 271 provide an auxiliary constraining force. The third area 18, the sixth area 28, the middle regions 172, 272 provide good ventilation.

The crotch section 5 is connected the first and second sheets 1, 2, and includes an absorbent body 51. The crotch section 5 is used to cover the user's crotch, and the absorbent body 51 is used to absorb and keep the user's urine and feces from leaking out.

Preferably, a width of the first and second notches 13, 23 measured in a transverse direction transverse to the longitudinal direction of the first and second sheets 1, 2 is one fourth to one half a width of the thermal seams (S) measured in the transverse direction. As such, the width of the first and second notches 13, 23 will not be too large to weaken the thermal seams (S) and will not make the thermal seams (s) easily separable. However, if the width of the first and second notches 13, 23 is too small, separation at the thermal seams (S) can become difficult.

Moreover, a width of the third and sixth areas 18, 28 measured in a transverse direction transverse to the longitudinal direction of the first and second sheets 1, 2 is preferably one fourth to one half a width of the first and second sheets 1, 2 measured in the transverse direction. As such, the width of the third and sixth areas 18, 28 will not be too large, and the areas for the elastic strings 41 will not be too small to offer sufficient constraining strength. However, if the width of the third and sixth areas 18, 28 is too small, the areas for the elastic strings 41 will be too large and will be unable to provide good ventilation.

The manufacturing process for the diaper 100 is illustrated as below. Firstly, the first and second sheets 1, 2 are spaced apart from each other, and the elastic strings 41 are attached to the first area 16, the second area 17, the fourth area 26 and the fifth area 27. In the second area 17 and the fifth area 27, the elastic strings 41 are adhered to the lateral regions 171, 271 using glue, parts of the elastic strings 41 lying over the middle regions 172, 272 are cut off so that the elastic strings 41 retract toward the lateral regions 171, 271, and the middle regions 172, 172 are free of the elastic strings 41. Subsequently, the first outermost edges 11 and the second outermost edges 21 are cut to form the first and second notches 13, 23, respectively. Thereafter, the crotch section 5 is attached to the first and second sheets 1, 2. Finally, the crotch section 5 is folded along a line (L) (as shown in FIG. 2), and the first and second margins 12, 22 are thermally bonded.

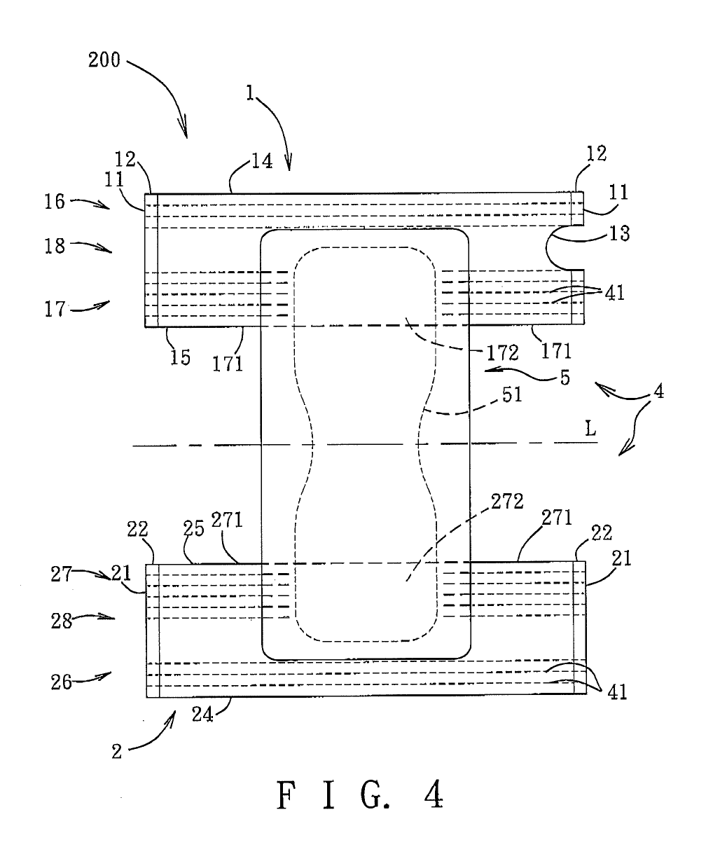

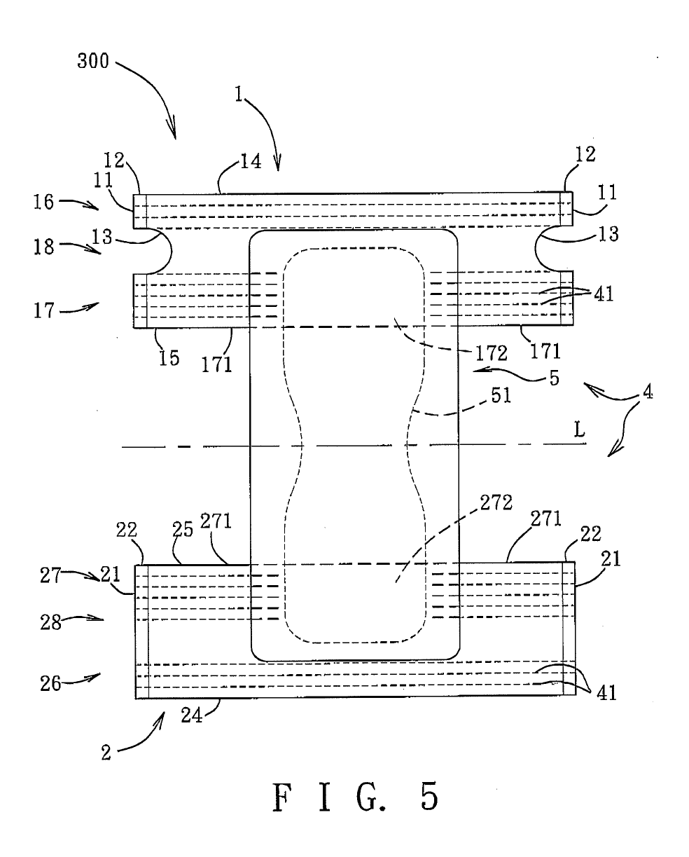

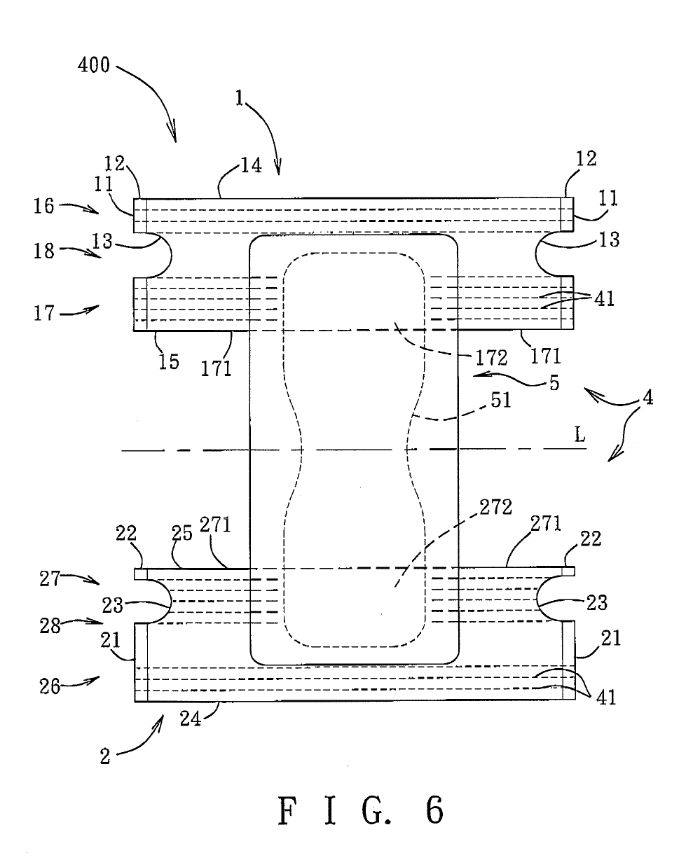

FIGS. 4 to 6 show diapers 100, 200, 300 according to the second, third and fourth preferred embodiments of the invention, which are substantially similar to the first preferred embodiment. However, the diaper 200 differs in that no second notches 23 are provided in the second sheet 2 and only one first notch 13 is in the first sheet 1. The diaper 300 differs in that the diaper 300 does not have the second notches 23. The diaper 400 differs in that the second notches 23 are formed in the fifth area 27.

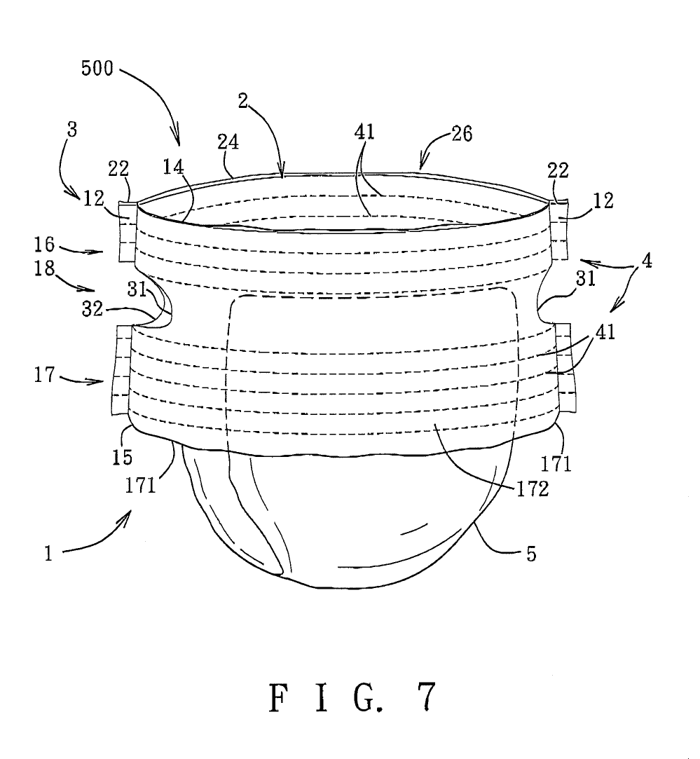

Referring to FIG. 7, the diaper 500 according to the fifth preferred embodiment differ from the diaper 100 in that the middle regions 172, 272 are provided with the elastic strings 41.

With the provision of the first notches 13 and the second notches 23, the bonding strength between the first margins 12 and the second margins 22 is reduced, and the diaper of the present invention can be removed easily from the wearer.

While the present invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A diaper comprising:
a longitudinal first sheet including two first outermost edges that are opposite in a longitudinal direction of said first sheet, two first margins each of which is contiguous to one of said first outermost edges, at least one first notch that is indented inwardly from one of said first outermost edges to extend inwardly through and beyond an adjacent one of said first margins, said first notch having a dimension in the longitudinal direction, which is larger than that of the adjacent one of said first margins;
a longitudinal second sheet including two second outermost edges that are opposite in a longitudinal direction of said second sheet, two second margins each of which is contiguous to one of said second outermost edges and each of which is in face-to-face contact with one of said first margins, said second margin being thermal bonded respectively to said first margins to form thermal seams so that said first and second sheets are formed into a waist-surrounding part, said first notch forming a first opening contiguous to one of said thermal seams;
elastic strings disposed in said first and second sheets; and
a crotch section having an absorbent body and connected to said first and second sheets.

2. The diaper as claimed in claim 1, wherein said first sheet includes two said first notches which are indented inwardly from said first outermost edges, respectively, said first notches forming said first openings contiguous to said thermal seams, respectively.

3. The diaper as claimed in claim 2, wherein said second sheet further includes two second notches each of which is indented inwardly from one of said second outermost edges to extend inwardly through and beyond an adjacent one of said second margins and each of which has a dimension in the longitudinal direction of said second sheet, which is larger than that of the adjacent one of said second margins, said second notches forming second openings contiguous to said thermal seams, respectively.

4. The diaper as claimed in claim 3, wherein said first notches are aligned respectively with said second notches, said first openings being communicated with said second openings, respectively.

5. The diaper as claimed in claim 1, wherein a width of said notch measured in a transverse direction transverse to the longitudinal direction of said first and second sheets is one fourth to one half a width of said thermal seams measured in the transverse direction.

6. The diaper as claimed in claim 1, wherein said first sheet further includes first and second lateral edges interconnecting said first outermost edges, a first area adjacent to said first lateral edge, a second area adjacent to said second lateral edge, and a third area interposed between and contiguous to said first and second areas, said second sheet further including third and fourth lateral edges interconnecting said second outermost edges, a fourth area adjacent to said third lateral edge, a fifth area adjacent to said fourth lateral edge, and a sixth area interposed between and contiguous to said fourth and fifth areas, said first, second, fourth and fifth areas having said elastic strings, said third and sixth areas being free of said elastic strings.

7. The diaper as claimed in claim 6, wherein a width of said third and sixth areas measured in a transverse direction transverse to the longitudinal direction of said first and second sheets is one fourth to one half a width of said first and second sheets measured in the transverse direction.

8. The diaper as claimed in claim 7, wherein said second area of said first sheet is divided into two lateral regions respectively contiguous to said first outermost edges, and a middle region disposed between and contiguous to said lateral regions, said fifth area of said second sheet being divided into two lateral regions respectively contiguous to said second outermost edges, and a middle region disposed between and contiguous to said lateral regions of said second sheet, said lateral regions of said first and second sheets having said elastic strings, said middle regions of said first and second sheets being free of said elastic strings.

9. The diaper as claimed in claim 8, wherein said elastic strings extend along the longitudinal direction of said first and second sheets.

10. The diaper as claimed in claim 7, wherein said first notch is disposed in said third area.
